(19) United States
(12) Patent Application Publication
(10) Pub. No.: US 2025/0093367 A1
(43) Pub. Date: Mar. 20, 2025

(54) QUANTIFICATION OF LOW AMOUNTS OF ANTIBODY SIDE-PRODUCTS

(71) Applicant: Hoffmann-La Roche Inc., Little Falls, NJ (US)

(72) Inventors: Laurent LARIVIERE, Westendorf (DE); Tilman SCHLOTHAUER, Penzberg (DE); Christian SPICK, Seeshaupt (DE); Adrian ZWICK, Iffeldorf (DE)

(73) Assignee: Hoffmann-La Roche Inc., Little Falls, NJ (US)

(21) Appl. No.: 18/672,669

(22) Filed: May 23, 2024

Related U.S. Application Data
(63) Continuation of application No. PCT/EP2022/082453, filed on Nov. 18, 2022.

(30) Foreign Application Priority Data
Nov. 25, 2021 (EP) 21210551.4

(51) Int. Cl. G01N 33/68; G01N 21/59; G01N 33/543

(52) U.S. Cl. CPC G01N 33/6857; G01N 21/59; G01N 33/54306; G01N 2021/5903

(57) ABSTRACT

The current invention is directed to a method for determining homodimeric avid-binding side-products of a bispecific antibody in a sample comprising the correctly assemble heterodimeric affine-binding bispecific antibody and the mis-assembled homodimeric avid-binding side-product of the bispecific antibody using surface plasmon resonance, wherein the correctly assembled heterodimeric affine-binding bispecific antibody comprises one or more binding site for a first antigen and one or more binding sites for a second antigen, wherein the mis-assembled homodimeric avid-binding side-product of the bispecific antibody comprises two or more binding sites to the first antigen but at least more than the correctly assembled bispecific antibody, wherein the correctly assembled bispecific antibody is a heterodimer and the mis-assembled bispecific antibody is a homodimer, wherein the presence of the homodimeric avid-binding side-product is determined if residual binding, i.e. an increased SPR signal, can be determined in the dissolution phase of the SPR analysis.

### CROSS-REFERENCE TO RELATED APPLICATIONS

This application is a Continuation of International Patent Application No. PCT/EP2022/082453, filed Nov. 18, 2022, which claims benefit of priority to EP patent application Ser. No. 21/210,551.4 filed Nov. 25, 2021, each of which is incorporated herein by reference in its entirety.

The current invention is in the field of analytical methods. The current invention is directed to a method for determining and quantifying antibody-related side-products resulting from wrong pairing of antibody chains, especially resulting in unwanted increased avid-binding properties of the side-product, for example of heteromeric multispecific antibodies. Such side-products have increased biological activity and can result in unwanted side effects compared to the correctly assembled multispecific antibody.

### BACKGROUND OF THE INVENTION

Surface plasmon resonance (SPR) is an optical technique utilized for detecting molecular interactions. Binding of a mobile molecule (analyte) to a molecule immobilized on a thin metal film (ligand) changes the refractive index of the film. The angle of extinction of light, reflected after polarized light impinges upon the film, is altered, monitored as a change in detector position for the dip in reflected intensity (the surface plasmon resonance phenomenon). Because the method strictly detects mass, there is no need to label the interacting components, thus eliminating possible changes of their molecular properties (Drescher, D. G., et al. Meth. Mol. Biol. 493 (2009) 323-343).

SPR binding analysis methodology is used to study molecular interactions. One interaction partner is immobilized directly by a chemical reaction, e.g. an amine-coupling reaction, or indirectly by forming a binding pair, e.g. biotin-streptavidin, on a sensor chip. The chip is then inserted into the flow chamber of an SPR instrument, e.g. available from BIAcore (BIAcore, Uppsala, Sweden). Addition of the second interaction partner, the flow-through analyte, to the chamber, results in binding thereof to the immobilized first interaction partner, producing a small change in refractive index at the gold surface. This change can be quantified with high precision. Binding affinities can be obtained from the ratio of rate constants, yielding a straightforward characterization of protein-protein interaction (Drescher, D. G., et al. Meth. Mol. Biol. 493 (2009) 323-343).

Unfortunately, the universal nature of SPR detection also constitutes a fundamental limitation: since every binding event causes a similar refractive index signal, it is not possible to discriminate different binding events resulting in similar mass changes. One area of rapidly growing interest where the information content of conventional SPR may be limited is in the study of bispecific binders (e.g. bispecific antibodies). Here, specialized assay formats are required, and the binding of the two species must be studied in sequence, rather than in parallel. A related case is that of competition-based assays where analyte molecules bind the surface species at the same or similar binding sites. In principle, competition assays can be performed using SPR by calculating residual binding. However, this is non-trivial for small molecule competitors as minor concentration differences in organic solvent vehicle (i.e. DMSO) across samples can have a large negative impact on the achievable signal to noise ratio and interfere with the ability to accurately determine the residual binding signal (Eng, L., et al., Biochem. Biophys. Res. Comm. 497 (2018) 133-138).

Eng et al. (supra) reported a Label-Enhanced SPR (LE-SPR) technique to increases the information content of SPR by enabling qualitative discrimination and quantitative monitoring of two different, simultaneously binding species using standard, commercial single-wavelength SPR instruments. LE-SPR is based on labeling of a binding molecule with a specialized dye label in combination with readout and software-based shape analysis of the entire SPR dip during the binding reaction. They have shown three examples of dual species detection: i) the accurate quantitative determination of two different species in mixed bulk solution samples; ii) the qualitative deconstruction of a conventional sensorgram of two different, simultaneously binding species into separate binding curves of the two species, followed by the faithful reconstruction of the original sensorgram; and iii) the simultaneous determination of binding dissociation constants of two different species in a mixture binding to a protein on the surface.

T-cell-dependent bispecific antibodies (TDBs or TCBs) are promising cancer immunotherapies that recruit a patient's T-cells to kill cancer cells. There are increasing numbers of TBDs in clinical trials, demonstrating their widely recognized therapeutic potential. Due to the fact that TDBs engage and activate T-cells via an anti-CD3 (aCD3) arm, aCD3 homodimer (aCD3 HD) and high-molecular-weight species (HMWS) are product-related impurities that pose a potential safety risk by triggering off-target T-cell activation through bivalent engagement and dimerization of T-cell receptors (TCRs). This off-target (also known as target-cell-independent) T-cell activation can induce cytokine secretion, triggering an undesired immune response in patients. Off-target T-cell activation by aCD3 HD is an undesired activity and distinct from the T-cell activation by TDB, which eventually kills the target tumor cells. Therefore, it is important to control the level of aCD3 HD in TDB drug products, and sensitive assays are needed to enable its quantification (Lee, H. Y., et al., Nature Sci. Rep. 9 (2019) 3900).

To monitor and control the level of non-specific T-cell activation, Lee et al. (supra) reported a sensitive and quantitative cell-based T-cell-activation assay, which can detect aCD3 HD in TDB drug product by exploiting its ability to activate T-cells in the absence of target cells.

WO 2020/200941 reported a heterodimeric fusion polypeptide comprising a first proteinaceous moiety and a second proteinaceous moiety, wherein the first proteinaceous moiety and the second proteinaceous moiety are the first and the second antigen of a bispecific antibody which comprises a first binding site that specifically binds to the first proteinaceous moiety and a second binding site that specifically binds to the second proteinaceous moiety, wherein the first proteinaceous moiety is fused to the N-terminus of a first antibody heavy chain Fc-region polypeptide of the IgG1 subtype, wherein the second proteinaceous moiety is fused to the N-terminus of a second antibody heavy chain Fc-region polypeptide of the IgG1 subtype, wherein the first and the second heavy chain Fc-region polypeptide form a disulfide-linked heterodimer, wherein one or both of the heavy chain Fc-region polypeptides comprise a tag for immobilization to a solid phase at its C-terminus, and wherein the first and the second Fc-region polypeptide comprise the mutations T366W and T366S/L368A/Y407V, respectively, and the use of said fusion polypeptide for the determination of the avidity-based binding strength of a bispecific antibody, which comprises a first binding site specifically binding to a first antigen and a second binding site specifically binding to a second antigen, to said first and second antigen in a surface-plasmon-resonance-method.

Amaral, M., et al. reported engineered technologies and bioanalysis of multispecific antibody formats (J. Appl. Bioanal. 2020 (6) 26-51).

US 2017/003295 reported SPR-based bridging assay format for determining the biological activity of multivalent, multispecific molecules.

WO 2014/177460 reported human FcRn-binding modified antibodies and methods of use.

JP 2020/202848 reported modified antigen-binding polypeptide constructs and uses thereof.

### SUMMARY OF THE INVENTION

Herein is reported a method for the determination or/and quantification of therapeutic antibody-related side-products based on the difference in the apparent binding affinity, i.e. based on an increase in binding avidity, between the correctly assembled therapeutic antibody and the therapeutic antibody-related side-product.

Without being bound by this theory, it is assumed that any antibody-related side-product that has an increased binding valency compared to the correctly assembled antibody can be determined or/and quantified using the approach as reported herein. Likewise, any method suitable for the determination of a difference in the apparent binding affinity can be used, such as, for example, ELISA and SPR.

An antibody-related side-product with increased apparent binding avidity comprises more binding sites to the respective target (i.e. antigen) than the correctly assembled antibody, e.g. two binding sites instead of one, four binding sites instead of two, etc.

The assay principles described herein can be applied to different bioassays using different detection methods, such as ELISA (enzyme-linked immunosorbent assay), SPR (surface plasmon resonance) or BLI (bio-layer interferometry).

For example, the difference in apparent affinities resulting from an increase in binding avidity between antibody and valency-increased antibody-related side-product(s) can be quantified in an ELISA. Therein, by applying appropriate washing conditions antibody-ligand interactions are dissolved while leaving the side-product-ligand complexes intact. Then, resulting signals will be proportional to the content of multivalent side-product(s) in the sample. The amount can be quantified by utilization of a calibration curve.

Likewise, SPR can be used to leverage side-product specific interaction properties resulting in different dissolution kinetics resulting in a time-dependent difference in the residual binding signal.

In more detail, in one aspect the current invention is directed to an SPR-based assay that allows determining (bispecific) antibody-related side-product quantities that were previously below the detection limit of established quantification methods.

The method according to the current invention is especially suited for the determination or/and quantification of antibody-related side-product(s) that have an increased binding valency to a target.

For the formation of bispecific antibodies, different approaches for fostering heterodimer formation are known. One is the knob-into-hole design (see, e.g., WO 96/027011, Ridgway, J. B., et al., Protein Eng. 9 (1996) 617-621; Merchant, A. M., et al., Nat. Biotechnol. 16 (1998) 677-681; WO 98/050431). During the development of bispecific antibodies, usually the binder with the higher potential for generating adverse events is linked to the knob-chain in a knob-into-hole antibody format. The formation of knob-knob homodimers is sterically disfavored compared to the formation of hole-hole homodimers. This leads to very low amounts of mis-paired knob-knob molecules as antibody-related side-product(s) during antibody production as this antibody composition is disfavored.

Although these knob-knob side-products compose a very small fraction of the antibody product there is a need to monitor them intensively as they are capable of causing adverse side-effects, e.g. toxicological, or reduce efficiency of the drug, e.g. by being unable to perform the intended biological action. These unwanted events are caused by enhanced drug-target complex stability due to avidity (avid binding).

The invention is based, at least in part, on the finding that such an avidity increase can be leveraged in an SPR assay to detect side-product concentrations that are lower than previously detectable by established methods such as SEC or MS. The established methods are using residual binding with respect to the isolated drug whereas the method according to the current invention uses an enrichment due to avidity, which is not used in other quantification methods.

In the SPR method according to the invention, the antibody target, i.e. the antigen, is immobilized on the SPR sensor chip. Thereafter the sample comprising the antibody and the antibody-related side-products to be detected is injected. This results in antibody-target complex formation on the sensor surface. After that, a buffer without antibody and also without antibody-related side-product is injected into the system so that the antibody-target complexes can dissociate. Thereby a dissociation profile is generated wherein the dissociation of the antibody-related side-product is slower compared to that of the correctly assembled antibody. Such slower dissociation results in an increased SPR binding signal after defined dissociation times compared to an antibody sample without antibody-related avid-binding side-products. This signal increase is the residual binding due to the avidity of the antibody-related homodimeric side-product(s). Thereby it is possible to quantify the amount of antibody-related homodimeric avid-binding side-product(s) present in the sample and thereby, finally, the concentration of antibody-related avid-binding homodimeric side-product(s) with a very low detection limit.

The current invention is based, at least in part, on the finding that the antibody target (=antigen) has to be immobilized with minimum density or in a density range depending on the format of the multispecific, e.g. bispecific, antibody to be tested. In certain embodiments, the antibody target has to be immobilized with at least 1000 RU. In one preferred embodiment, the target is immobilized with at least 2000 RU. In certain embodiments, the target is immobilized with at least 4000 RU. In certain embodiments, the target is immobilized with 2000-4000 RU.

The invention is based, at least in part, on the finding that the detection limit of the method can be lowered by prolonging the antibody injection time, as the antibody-related side-products that are capable of avid binding will accumulate on the chip surface during the association time. In certain embodiments, the association time is at least 240 seconds. In certain embodiments, the association time is at least 300 seconds. In one preferred embodiment, the injection time is at least 360 seconds. In certain embodiments, the association time is at least 420 seconds.

The current invention is based, at least in part, on the finding that the difference between antibody complex stability and antibody-related side-product complex stability on an SPR chip can be used to quantify the antibody-related side-product. In one preferred embodiment, the dissociation time at that the residual binding is determined is at least 1200 seconds. In certain embodiments, the dissociation time at that the residual binding is determined is at least 1800 seconds.

The residual binding is depending on the amount or fraction of antibody-related avid-binding side-product in the sample as well as the complex stability of the antibody-related side-product with the target, i.e. the difference in complex stability compared to the correctly assembled antibody.

Thus, in case the residual binding is small the assay conditions can be adjusted to increase the residual binding. This can be done in knowledge of the current invention by different measures known to a person skilled in the art based on general knowledge, e.g. about the thermodynamic and kinetic properties of the isolated antibody and the antibody-related side-product to be detected. Exemplary measures are listed in the following table.

| Measure | Result |
|---|---|
| prolonged | prolonging the association phase increases |
| association | the amount of accumulated avidity-driven |
| | complexes. |
| prolonged | in case of stable avid binding, prolonging |
| dissociation | the dissociation can be sufficient to dissolve |
| | all monovalent antibody-target complexes |
| buffer salt | destabilization of the complex is more |
| concentration | pronounced for affinity-driven than for |
| increase | avidity-driven complexes |
| buffer pH change | destabilization of the complex is more |
| | pronounced for affinity-driven than for |
| | avidity-driven complexes |
| assay temperature | destabilization of the complex is more |
| increase | pronounced for affinity-driven than for |
| | avidity-driven complexes |

One aspect according to the current invention is a method for determining a mis-assembled side-product of an antibody in a sample comprising the correctly assembled antibody and the mis-assembled side-product of the antibody,
wherein the correctly assembled antibody and the mis-assembled side-product of the antibody bind to a (first) target,
wherein the mis-assembled side-product of the antibody comprises more binding sites to the target than the correctly assembled antibody,
the method comprising the following steps:
   i) immobilizing the target on the surface of an SPR chip at high density, preferably at least with 1000 RU, more preferably with at least 1500 RU, most preferably with at least 2000 RU,
   ii) applying a buffered solution comprising the correctly-assembled antibody (as sole species binding to the target) to the surface obtained in step i) to generate a loaded surface, preferably for at least 5 min., more preferably for at least 7 min, most preferably for at least 10 min, preferably at a flow rate of 30 μL/min or less/in the range of 50 μL/min to 5 μL/min, more preferably of 25 μL/min or less/in the range of 40 μL/min to 7.5 L/min, most preferably of 20 μL/min or less/in the range of 30 μL/min to 10 μL/min,
   iii) applying a (buffered) solution not comprising a compound binding to the target (i.e. not comprising the correctly-assembled antibody or the mis-assembled side-product of the antibody) to the loaded surface obtained in step ii), preferably for at least 7.5 min., more preferably for at least 15 min, most preferably for at least 20 min, and recording the decay of the SPR signal,
   iv) optionally regenerating the SPR surface,
   v) applying a (buffered) sample solution suspected to comprise the mis-assembled side-product of the antibody to the an SPR surface obtained analogously as in step i) or to the regenerated SPR surface of step iv) to generate a loaded surface, preferably for at least 5 min., more preferably for at least 7 min, most preferably for at least 10 min, preferably at a flow rate of 30 μL/min or less/in the range of 50 μL/min to 5 μL/min, more preferably of 25 μL/min or less/in the range of 40 μL/min to 7.5 μL/min, most preferably of 20 μL/min or less/in the range of 30 μL/min to 10 μL/min,
   vi) applying a (buffered) solution not comprising a compound binding to the target to the loaded surface obtained in step v), preferably for at least 7.5 min., more preferably for at least 15 min, most preferably for at least 20 min, and recording the decay of the SPR signal,
   vii) determining the mis-assembled side-product of the antibody if the SPR signal determined in step vi) is/remains above the SPR signal determined in step iii) after the start of the application of the (buffered) solution not comprising a compound binding to the target, preferably at least 7.5 min. after the start, more preferably at least 15 min. after the start, most preferably 20 min. after the start of applying a (buffered) solution not comprising a compound binding to the target.

One aspect according to the current invention is a method for determining a mis-assembled side-product of a bispecific antibody in a sample comprising the correctly assemble bispecific antibody and the mis-assembled side-product of the bispecific antibody using surface plasmon resonance,
wherein the correctly assembled bispecific antibody comprises exactly one binding site for a first antigen and one or more binding sites for a second antigen, i.e. the bispecific antibody has one binding site (specifically) binding to a first antigen and one or more binding sites (specifically) binding to a second antigen,
wherein the mis-assembled side-product of the bispecific antibody comprises two or more binding sites to the first antigen and zero to two binding sites for the second antigen,
wherein the correctly assembled bispecific antibody is a heterodimer and the mis-assembled bispecific antibody is a homodimer with respect to the Fc-region, preferably the heterodimer is formed by the knobs-into-hole method,
the method comprising the following steps:
   i) immobilizing the first antigen on the surface of at least one flow-cell of an SPR chip at high density, preferably at least with 1000 RU, more preferably with at least 1500 RU, most preferably with at least 2000 RU,
   ii) applying a buffered solution comprising the isolated bispecific antibody as sole species binding to the antigen (or a reference standard comprising the correctly assembled bispecific antibody and an upper limit amount of the mis-assembled side-product of the bispecific antibody) to the at least one flow-cell obtained in step i), preferably for at least 5 min., more preferably for at least 7 min, most preferably for at least 10 min, preferably at a flow rate of 30 μL/min or less/in the range of 50 μL/min to 5 μL/min, more preferably of 25 μL/min or less/in the range of 40 μL/min to 7.5 μL/min, most preferably of 20 μL/min or less/in the range of 30 μL/min to 10 μL/min,
   iii) applying a buffered solution not comprising a compound binding to the antigen (i.e. not comprising the bispecific antibody or the mis-assembled side-product of the bispecific antibody) to the at least one flow-cell obtained in step ii), preferably for at least 7.5 min., more preferably for at least 15 min, most preferably for at least 20 min, and recording the decay of the SPR signal, and optionally thereafter regenerating the at least one flow cell,
   iv) applying a buffered sample solution suspected to comprise the mis-assembled side-product of the bispecific antibody (or an amount of the mis-assembled side-product of the bispecific antibody above the upper limit amount) to at least one flow-cell obtained analogously as in step i) or the regenerated flow-cell obtained in step iii), preferably for at least 5 min., more preferably for at least 7 min, most preferably for at least 10 min, preferably at a flow rate of 30 μL/min or less/in the range of 50 μL/min to 5 L/min, more preferably of 25 μL/min or less/in the range of 40 L/min to 7.5 μL/min, most preferably of 20 μL/min or less/in the range of 30 μL/min to 10 μL/min,
   v) applying a buffered solution not comprising a compound binding to the antigen (i.e. not comprising the bispecific antibody or the mis-assembled side-product of the bispecific antibody) to the at least one flow-cell obtained in step
   iv), preferably for at least 7.5 min., more preferably for at least 15 min, most preferably for at least 20 min, and recording the decay of the SPR signal,
   vi) determining the mis-assembled side-product of the bispecific antibody if the SPR signal determined in step v) is above the SPR signal determined in step iii) after the start of the application of the buffered solution not comprising a compound binding to the antigen (i.e. not comprising the bispecific antibody or the mis-assembled side-product of the bispecific antibody), preferably at least 7.5 min. after the start, more preferably at least 15 min. after the start, most preferably 20 min. after the start of the applying a (buffered) solution not comprising a compound binding to the target.

One aspect according to the current invention is a method for determining avid-binding side-products of a bispecific antibody in a sample comprising correctly assembled bispecific antibody and one or more avid-binding side-product of the bispecific antibody using surface plasmon resonance,
wherein the correctly assembled bispecific antibody comprises one or more binding site for a first antigen and one or more binding sites for a second antigen,
wherein the avid-binding side-product of the bispecific antibody comprises two or more binding sites to the first antigen and more binding sites to the first antigen than the correctly assembled bispecific antibody,
the method comprising the following steps:
   i) applying a loading solution comprising the correctly assembled bispecific antibody to an SPR surface on which the first antigen has been immobilized with at least 1000 RU to generate an SPR signal, applying thereafter a dissolution solution not comprising a compound binding to the first antigen to the SPR chip and recording the decay of the SPR signal,
   ii) applying a sample solution suspected to comprise an avid-binding side-product of the bispecific antibody to an SPR surface on which the first antigen has been immobilized with at least 1000 RU to generate an SPR signal, applying thereafter a dissolution solution not comprising a compound binding to the first antigen to the SPR chip and recording the decay of the SPR signal,
   iii) determining the avid-binding side-product of the bispecific antibody if the decay of the SPR signal determined in step ii) is slower than the decay of the SPR signal determined in step i).

### DETAILED DESCRIPTION OF EMBODIMENTS OF THE INVENTION

The current invention is directed to a method for determining homodimeric avid-binding side-products of a bispecific antibody in a sample comprising the correctly assemble heterodimeric affine-binding bispecific antibody and the mis-assembled homodimeric avid-binding side-product of the bispecific antibody using surface plasmon resonance, wherein the correctly assembled heterodimeric affine-binding bispecific antibody comprises one or more binding site for a first antigen and one or more binding sites for a second antigen, wherein the mis-assembled homodimeric avid-binding side-product of the bispecific antibody comprises two or more binding sites to the first antigen but at least more than the correctly assembled bispecific antibody, wherein the correctly assembled bispecific antibody is a heterodimer and the mis-assembled bispecific antibody is a homodimer, wherein the presence of the homodimeric avid-binding side-product is determined if residual binding, i.e. an increased SPR signal, can be determined in the dissolution phase of the SPR analysis.

The invention is based, at least in part, on the finding that an avidity increase due to mis-assembly of a multispecific antibody can be leveraged in an SPR assay to detect side-product concentrations that are lower than previously detectable by established methods such as SEC or MS. The established methods are using residual binding with respect to the isolated drug whereas the method according to the current invention uses an enrichment due to avidity, which is not used by other quantification methods.

Thus, the current invention is directed to a method for the determination or/and quantification of therapeutic antibody-related side-products based on the difference in the apparent binding affinity, i.e. based on an increase in binding avidity, between the correctly assembled therapeutic antibody and the therapeutic antibody-related side-product.

The method according to the current invention allows the determination of the presence of antibody-related side-products that have an increased binding valency compared to the correctly assembled antibody.

With other words, an antibody-related side-product with increased apparent binding avidity comprises more binding sites to the respective target (i.e. antigen) than the correctly assembled antibody, e.g. two binding sites instead of one, four binding sites instead of two, etc.

### Definitions

The term “about” denotes a range of +/−20% of the thereafter following numerical value. In certain embodiments, the term “about” denotes a range of +/−10% of the thereafter following numerical value. In certain embodiments, the term “about” denotes a range of +/−5% of the thereafter following numerical value.

“Affinity” or “binding affinity” refers to the strength of the sum total of non-covalent interactions between a single binding site of a molecule (e.g. an antibody) and its binding partner (=target) (e.g. an antigen). Unless indicated otherwise, as used herein, “binding affinity” refers to intrinsic binding affinity that reflects a 1:1 interaction between members of a binding pair (e.g. antibody and target (antigen)). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

The term “antibody” herein is used to define at least heterodimeric multispecific antibodies (e.g. bispecific antibodies, trispecific antibodies).

A Y-shaped full-length antibody in general comprises two so-called light chain polypeptides (light chains) and two so-called heavy chain polypeptides (heavy chains). Each of the heavy and light chain polypeptides contains a variable domain (variable region) (generally the amino terminal portion of the polypeptide chain) comprising binding regions that are able to interact with an antigen. A pair of a light chain variable domain and a heavy chain variable domain forms a binding site of the antibody. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The constant domains of an antibody heavy chain comprise the CH1-domain, the CH2-domain and the CH3-domain, whereas the light chain comprises only one constant domain, CL, which can be of the kappa isotype or the lambda isotype.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “binding (to an antigen)” denotes the binding of an antibody to its antigen in an in vitro assay, in certain embodiments in a binding assay in which the target is bound to a surface and binding of the antibody to the target is measured by Surface Plasmon Resonance (SPR). Binding means the measurement of the binding capacity of e.g. the antibody for target A or target B, or for a capture molecule e.g. anti-human-Fab-capture for the antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “valent” as used within the current application denotes the presence of a specified number of binding sites in an antibody. As such, the terms “bivalent”, “tetravalent”, and “hexavalent” denote the presence of two binding sites, four binding sites, and six binding sites, respectively, in an antibody. A bispecific antibody, for example, comprises at least two binding sites whereby the binding sites specifically bind to different targets.

The term “binding affinity” denotes the strength of the interaction of a single binding site with its respective target. Experimentally, the affinity can be determined e.g. by measuring the kinetic constants for association (kA) and dissociation (kD) of the antibody and the antigen in the equilibrium.

The term “binding avidity” denotes the combined strength of the interaction of multiple binding sites of one antibody with the same target. As such, avidity is the combined synergistic strength of bond affinities rather than the sum of bonds. Requisites for avidity are a polyvalence of an antibody or of a functional multimer to one target (antigen), multiple accessible epitopes on one soluble target, or multiple binding of an antibody to one epitope each on various immobilized targets. Thus, such an antibody shows avid-binding to its target.

In principle, the complex association does not differ between affine and avid binding. However, the complex dissociation in case of avid binding depends on the simultaneous dissociation of all binding sites involved. Therefore, the increase of binding strength due to avid binding (compared to affine binding) depends on the dissociation kinetics/complex stability. Thereby, for example, a monovalent binder will dissociate faster in comparison to a multivalent binder. The enrichment of the multivalent binding over extended periods of time according to the method of the invention can be used for increasing the sensitivity of the method.

An “isolated” antibody is one that has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

### Multispecific Antibodies

In the method according to the current invention, the amount of avid-binding side-product(s) of an at least heterodimeric and at least bispecific antibody is determined.

Multispecific antibodies are monoclonal antibodies that have binding specificities for at least two different targets, i.e. antigens. In certain embodiments, one of the binding specificities is for a first antigen and the other is for a different second antigen. In certain embodiments, multispecific antibodies bind to two different epitopes of the same antigen.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004) or using leucine zippers to produce bi-specific antibodies (see, e.g., Kostelny, S. A., et al., J. Immunol. 148 (1992) 1547-1553.

The antibody can also be a multispecific antibody as described in WO 2009/080251, WO 2009/080252, WO 2009/080253, WO 2009/080254, WO 2010/112193, WO 2010/115589, WO 2010/136172, WO 2010/145792, or WO 2010/145793.

Bispecific antibodies are generally antibody molecules that specifically bind to two different, non-overlapping epitopes on the same antigen or to two epitopes on different antigens.

Different bispecific antibody formats are known.

Exemplary bispecific antibody formats for which the method according to the invention can be used are
the CrossMab format (=CrossMab): a multispecific IgG antibody comprising a first Fab fragment and a second Fab fragment, wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain);
   b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains are replaced by each other and the VH and VL domains are replaced by each other (i.e. the light chain of the first Fab fragment comprises a VH and a CH1 domain and the heavy chain of the first Fab fragment comprises a VL and a CL domain); and
wherein the second Fab fragment comprises a light chain comprising a VL and a CL domain, and a heavy chain comprising a VH and a CH1 domain;
the CrossMab may comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain, e.g. as disclosed in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, or WO 2013/096291 (incorporated herein by reference);
the one-armed single chain format (=one-armed single chain antibody): antibody comprising a first binding site that specifically binds to a first epitope or antigen and a second binding site that specifically binds to a second epitope or antigen, whereby the individual chains are as follows
   light chain (variable light chain domain+light chain kappa constant domain)
   combined light/heavy chain (variable light chain domain+light chain constant domain+peptidic linker+variable heavy chain domain+CH1+Hinge+CH2+CH3 with knob mutation)
   heavy chain (variable heavy chain domain+CH1+Hinge+CH2+CH3 with hole mutation);
the T-cell bispecific format (TCB format): a multispecific IgG antibody comprising
a) a first and a second Fab fragment that each specifically bind to a first antigen,
b) one domain exchanged Fab fragment that specifically binds to a second antigen in which the CH1 and the CL domain are exchanged for each other,
c) one Fc-region comprising a first heavy chain Fc-region polypeptide and a second heavy chain Fc-region polypeptide,
wherein the C-terminus of CH1 domain of the first Fab fragment is connected to the N-terminus of one of the heavy chain Fc-region polypeptides and the C-terminus of the CL-domain of the domain exchanged Fab fragment is connected to the N-terminus of the other heavy chain Fc-region polypeptide, and
wherein the C-terminus of the CH1 domain of the second Fab fragment is connected to the N-terminus of the VH domain of the first Fab fragment or to the N-terminus of the VH domain of the domain exchanged Fab fragment, and
wherein the first antigen or the second antigen is human CD3;
or comprising
a) a first and a second Fab fragment that each specifically bind to a first antigen,
b) one domain exchanged Fab fragment that specifically binds to a second antigen in which the VH and the VL domain are exchanged for each other,
c) one Fc-region comprising a first heavy chain Fc-region polypeptide and a second heavy chain Fc-region polypeptide,
wherein the C-terminus of CH1 domain of the first Fab fragment is connected to the N-terminus of one of the heavy chain Fc-region polypeptides and the C-terminus of the CH1-domain of the domain exchanged Fab fragment is connected to the N-terminus of the other heavy chain Fc-region polypeptide, and
wherein the C-terminus of the CH1 domain of the second Fab fragment is connected to the N-terminus of the VH domain of the first Fab fragment or to the N-terminus of the VL domain of the domain exchanged Fab fragment, and
wherein the first antigen or the second antigen is human CD3;
full-length antibody with domain exchange and additional heavy chain C-terminal binding site (2+1-format): a multispecific IgG antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein the additional Fab fragment specifically binding to the second antigen i) comprises a domain crossover such that a) the light chain variable domain (VL) and the heavy chain variable domain (VH) are replaced by each other, or b) the light chain constant domain (CL) and the heavy chain constant domain (CH1) are replaced by each other, or ii) is a single chain Fab fragment;
the two-armed single chain format (=two-armed single chain antibody): antibody comprising a first binding site that specifically binds to a first epitope or antigen and a second binding site that specifically binds to a second epitope or antigen, whereby the individual chains are as follows
   combined light/heavy chain 1 (variable light chain domain+light chain constant domain+peptidic linker+variable heavy chain domain+CH1+Hinge+CH2+CH3 with hole mutation)
   combined light/heavy chain 2 (variable light chain domain+light chain constant domain+peptidic linker+variable heavy chain domain+CH1+Hinge+CH2+CH3 with knob mutation);
the common light chain bispecific format (=common light chain bispecific antibody): antibody comprising a first binding site that specifically binds to a first epitope or antigen and a second binding site that specifically binds to a second epitope or antigen, whereby the individual chains are as follows
   light chain (variable light chain domain+light chain constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation).

The term “TCB”, as used herein, denotes a T-cell bispecific antibody. Such an antibody can have a format as described, e.g. in WO 2013/026831. Those molecules can simultaneously bind to CD3 (first specificity) on T-cells and to an antigen on a target (e.g. tumor) cell (second specificity) and thereby induce killing of target cells. TCB are trivalent bispecific antibodies consisting of four polypeptides or polypeptide chains: one light chain, which is a full length light chain; a further light chain, which is a domain exchanged full length light chain; one heavy chain, which is a full length heavy chain; and a further heavy chain, which is an extended heavy chain comprising an addition domain exchanged heavy or light chain Fab fragment.

The term “domain crossover” as used herein denotes that in a pair of an antibody heavy chain VH-CH1 fragment and its corresponding cognate antibody light chain, i.e. in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH-CH1 heavy chain fragment (“Fab crossover”), which leads to by domain crossover to a light chain with a VH-CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W., et al, Proc. Natl. Acad. Sci USA 108 (2011) 11187-11192. Such antibodies are generally termed CrossMab.

In certain embodiments, the multi- or bispecific antibody is in the CrossMab-format.

In certain embodiments, the multi- or bispecific antibody is in the TCB-format.

In certain embodiments, the multi- or bispecific antibody is in the 2+1-format.

In certain embodiments, the multi- or bispecific antibody is a one-armed single chain antibody.

In certain embodiments, the multi- or bispecific antibody is a two-armed single chain antibody.

In certain embodiments, the multi- or bispecific antibody is a common light chain antibody.

### Heterodimerization

There exist several approaches for CH3-modifications to enforce the heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically in all such approaches the first CH3 domain and the second CH3 domains are both engineered in a complementary manner so that each CH3 domain (or the heavy chain comprising it) can no longer form homodimers with itself but is forced to heterodimerize with the complementary engineered other CH3 domain (so that the first and second CH3 domain heterodimerize and no homodimers between the two first or the two second CH3 domains are formed). These different approaches for improved heavy chain heterodimerization are contemplated as different alternatives in combination with the heavy-light chain modifications (VH and VL exchange/replacement in one binding arm and the introduction of substitutions of charged amino acids with opposite charges in the CH1/CL interface) in the multispecific antibodies according to the invention which reduce light chain mispairing an Bence-Jones type side products.

Exemplary mutations in the CH3 domains introduced to facilitate the formation of heterodimeric Fc-regions, e.g. for the production of multi- or bispecific antibodies are outlined in the following.

Examples of such mutations are the so-called “knobs into holes” substitutions (see, e.g., WO 96/027011, Ridgway, J. B., et al., Protein Eng. 9 (1996) 617-621; Merchant, A. M., et al., Nat. Biotechnol. 16 (1998) 677-681; WO 98/050431; U.S. Pat. No. 7,695,936 and US 2003/0078385). The following knobs and holes substitutions in the individual polypeptide chains of an Fc-region of an IgG antibody of subclass IgG1 have been found to increase heterodimer formation: 1) Y407T in one chain and T366Y in the other chain; 2) Y407A in one chain and T366W in the other chain; 3) F405A in one chain and T394W in the other chain; 4) F405W in one chain and T394S in the other chain; 5) Y407T in one chain and T366Y in the other chain; 6) T366Y and F405A in one chain and T394W and Y407T in the other chain; 7) T366W and F405W in one chain and T394S and Y407A in the other chain; 8) F405W and Y407A in one chain and T366W and T394S in the other chain; and 9) T366W in one heavy chain Fc-region and T366S, L368A, and Y407V in the respective other heavy chain Fc-region, whereby the last listed is especially suited and, thus, the subject of one preferred embodiment. In addition, substitutions allowing the formation of new disulfide bridges between the two Fc-region polypeptide chains facilitate heterodimer formation (see, e.g., Merchant, A. M., et al., Nature Biotech. 16 (1998) 677-681; Atwell, S., et al., J. Mol. Biol. 270 (1997) 26-35; US 2003/0078385). The following substitutions resulting in appropriately spaced apart cysteine residues for the formation of new intra-chain disulfide bonds in the individual polypeptide chains of an Fc-region of an IgG antibody of subclass IgG1 have been found to increase heterodimer formation: in one preferred embodiment Y349C in one heavy chain Fc-region and S354C in the respective other heavy chain Fc-region; Y349C in one chain and E356C in the other; Y349C in one chain and E357C in the other; L351C in one chain and S354C in the other; T394C in one chain and E397C in the other; or D399C in one chain and K392C in the other.

Further examples of heterodimerization facilitating amino acid changes are the so-called “charge pair substitutions” (see, e.g., WO 2009/089004). The following charge pair substitutions in the individual polypeptide chains of an Fc-region of an IgG antibody of subclass IgG1 have been found to increase heterodimer formation: 1) K409D or K409E in one chain and D399K or D399R in the other chain; 2) K392D or K392E in one chain and D399K or D399R in the other chain; 3) K439D or K439E in one chain and E356K or E356R in the other chain; 4) K370D or K370E in one chain and E357K or E357R in the other chain; 5) K409D and K360D in one chain plus D399K and E356K in the other chain; 6) K409D and K370D in one chain plus D399K and E357K in the other chain; 7) K409D and K392D in one chain plus D399K, E356K, and E357K in the other chain; 8) K409D and K392D in one chain and D399K in the other chain; 9) K409D and K392D in one chain and D399K and E356K in the other chain; 10) K409D and K392D in one chain and D399K and D357K in the other chain; 11) K409D and K370D in one chain and D399K and D357K in the other chain; 12) D399K in one chain and K409D and K360D in the other chain; and 13) K409D and K439D in one chain and D399K and E356K on the other.

In one preferred embodiment of all aspects and embodiment, the first Fc-region polypeptide comprises the mutations Y349C, T366S, L368A and Y407V (“hole”) and the second Fc-region polypeptide comprises the mutations S354C and T366W (“knob”).

Other techniques for CH3-modifications to enforcing the heterodimerization are contemplated as alternatives to be used in the method of the invention and described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

The heterodimerization approach described in WO 2013/157953 can be used alternatively. In certain embodiments, a first CH3 domain comprises amino acid T366K mutation and a second CH3 domain polypeptide comprises amino acid L351D mutation. In certain embodiments, the first CH3 domain comprises further amino acid L351K mutation. In certain embodiments, the second CH3 domain comprises further amino acid mutation selected from Y349E, Y349D and L368E (preferably L368E).

The heterodimerization approach described in WO 2012/058768 can be used alternatively. In certain embodiments, a first CH3 domain comprises amino acid L351Y, Y407A mutations and a second CH3 domain comprises amino acid T366A, K409F mutations. In certain embodiments, the second CH3 domain comprises a further amino acid mutation at position T411, D399, S400, F405, N390, or K392 e.g. selected from a) T411 N, T411 R, T411Q, T411 K, T411D, T411E or T411W, b) D399R, D399W, D399Y or D399K, c S400E, S400D, S400R, or S400K F4051, F405M, F405T, F405S, F405V or F405W N390R, N390K or N390D K392V, K392M, K392R, K392L, K392F or K392E. In certain embodiments, a first CH3 domain comprises amino acid L351Y, Y407A mutations and a second CH3 domain comprises amino acid T366V, K409F mutations. In certain embodiments, a first CH3 domain comprises amino acid Y407A mutations and a second CH3 domain comprises amino acid T366A, K409F mutations. In certain embodiments, the second CH3 domain comprises a further amino acid K392E, T411E, D399R and S400R mutations.

In certain embodiments, the heterodimerization approach described in WO 2011/143545 can be used alternatively e.g. with the amino acid modification at a position selected from the group consisting of 368 and 409.

The heterodimerization approach described in WO 2011/090762 that also uses the knobs-into-holes technology described above can be used alternatively. In certain embodiments, a first CH3 domain comprises amino acid T366W mutations and a second CH3 domain comprises amino acid Y407A mutations. In certain embodiments, a first CH3 domain comprises amino acid T366Y mutations and a second CH3 domain comprises amino acid Y407T mutations.

The heterodimerization approach described in WO 2009/089004 can be used alternatively. In certain embodiments, a first CH3 domain comprises amino acid substitution of K392 or N392 with a negative-charged amino acid (e.g. glutamic acid (E), or aspartic acid (D), preferably K392D or N392D) and a second CH3 domain comprises amino acid substitution of D399, E356, D356, or E357 with a positive-charged amino acid (e.g. Lysine (K) or arginine (R), preferably D399K, E356K, D356K, or E357K and in one preferred embodiment D399K and E356K. In certain embodiments, the first CH3 domain further comprises amino acid substitution of K409 or R409 with a negative-charged amino acid (e.g. glutamic acid (E), or aspartic acid (D), preferably K409D or R409D). In certain embodiments, the first CH3 domain further or alternatively comprises amino acid substitution of K439 and/or K370 with a negative-charged amino acid (e.g. glutamic acid (E), or aspartic acid (D)).

The heterodimerization approach described in WO 2007/147901 can be used alternatively. In certain embodiments, a first CH3 domain comprises amino acid K253E, D282K, and K322D mutations and a second CH3 domain comprises amino acid D239K, E240K, and K292D mutations.

In certain embodiments, the heterodimerization approach described in WO 2007/110205 can be used alternatively.

### Recombinant Methods and Compositions

Antibodies may be produced using recombinant methods and compositions, e.g., as described in U.S. Pat. No. 4,816,567. In certain embodiments, isolated nucleic acid(s) encoding an antibody is (are) provided. Such nucleic acid may encode an amino acid sequence comprising the VL and/or an amino acid sequence comprising the VH of the antibody (e.g., the light and/or heavy chains of the antibody). In certain embodiments, one or more plasmids (e.g., expression plasmids) comprising such nucleic acids are provided. In certain embodiments, a host cell comprising such nucleic acid is provided. In certain embodiments, the host cell comprises (e.g., has been transformed with): (1) a plasmid comprising a nucleic acid that encodes an amino acid sequence comprising the VL of the antibody and an amino acid sequence comprising the VH of the antibody, or (2) a first plasmid comprising a nucleic acid that encodes an amino acid sequence comprising the VL of the antibody and a second plasmid comprising a nucleic acid that encodes an amino acid sequence comprising the VH of the antibody. In certain embodiments, the host cell is eukaryotic, e.g. a Chinese Hamster Ovary (CHO) cell or lymphoid cell (e.g., Y0, NS0, Sp2/0 cell). In certain embodiments, the method according to the current invention further comprises making the antibody as tested therein, wherein the method comprises culturing a host cell comprising a nucleic acid encoding the antibody, as provided above, under conditions suitable for expression of the antibody, recovering the antibody from the host cell (or host cell culture medium) and subjecting the antibody to a method according to the current invention for measuring or determining the content of antibody-related avid-binding side-product(s).

For recombinant production of an antibody with a method as outlined above, nucleic acids encoding an antibody, e.g., as described above, are isolated and inserted into one or more plasmids for further cloning and/or expression in a host cell. Such nucleic acid may be readily isolated and sequenced using conventional procedures (e.g., by using oligonucleotide probes that are capable of binding specifically to genes encoding the variant Fc-region polypeptide or the heavy and light chains of the antibody).

Suitable host cells for cloning or expression of antibody-encoding plasmids include eukaryotic cells.

For example, eukaryotic microbes such as filamentous fungi or yeast are suitable cloning or expression hosts for antibody-encoding plasmids, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

For example, mammalian cell lines that are adapted to grow in suspension may be useful. Other examples of useful mammalian host cell lines are monkey kidney CV1 line transformed by SV40 (COS-7); human embryonic kidney line (HEK293 or 293 cells as described, e.g., in Graham, F. L., et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK) p; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian host cell lines include Chinese hamster ovary (CHO) cells, including DHFR-CHO cells (Urlaub, G., et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268. In one preferred embodiment, the host cell is a CHO or HEK cell.

### State of the Art Surface-Plasmon-Resonance Methods

Kinetic binding parameters of antibodies to their respective antigens can be investigated by surface plasmon resonance, e.g. using a BIAcore instrument (GE Healthcare Biosciences AB, Uppsala, Sweden).

Briefly, for affinity determination a capture agent, e.g. the target/antigen of an antibody is covalently immobilized on a chip, e.g. on a CM5 chip via amine coupling, for capture and presentation to the respective antibodies to be analyzed.

For example, about 2,000-12,000 response units (RU) of a 10-30 μg/ml target solution is coupled onto all flow cells of a CM5 sensor chip in a BIAcore T200 instrument at pH 5.0 at a flow rate of 10-30 μl/min, by using an amine coupling kit supplied by GE Healthcare.

Binding is measured in HBS buffer (HBS-P (10 mM HEPES, 150 mM NaCl, 0.005% Tween 20, pH 7.4), or HBS-EP+ (0.01 M HEPES, 0.15 M NaCl, 3 mM EDTA, 0.05% v/v Surfactant PS20, pH 7.4), or HBS-ET (10 mM HEPES pH 7.4, 150 mM NaCl, 3 mM EDTA, 0.005% w/v Tween 20)), or any other applicable buffer at 25° C. (or alternatively at a different temperature in the range from 4° C. to 45° C.).

Therefor the antibody is injected for 30 seconds with a concentration in the range of 10 nM to 1 μM and bound to the active flow cells (e.g. 2, 3, and 4 or 2 and 3) leaving the other flow cells as reference flow cells.

Then the corresponding antigens (secondary targets) are added in various concentrations in solution, such as e.g. 144 nM, 48 nM, 16 nM, 5.33 nM, 1.78 nM, 0.59 nM, 0.20 nM and 0 nM, depending on the affinity of the antibody.

Association is measured by an antigen injection.

Dissociation is measured by washing the chip surface with buffer not comprising the analyte.

A K_{D} value is estimated using a 1:1 Langmuir binding model using the manufacturer's software and instructions. Negative control data (e.g. buffer curves) are subtracted from sample curves for correction of system intrinsic baseline drift and for noise signal reduction.

### EXAMPLES AND EXEMPLARY EMBODIMENTS OF THE METHOD ACCORDING TO THE INVENTION

The Method According to the Invention with a 2+1-Format Antibody

The method according to the invention is in the following first Example exemplified with a bispecific anti-Abeta/transferrin receptor antibody. This is merely presented to exemplify the invention and shall not be construed as limitation. The true scope of the invention is set forth in the appended claims.

The bispecific anti-Abeta/transferrin receptor antibody used in this example is formed of a Y-shaped full length antibody comprising two binding sites specifically binding to human Abeta protein and one additional monospecific Fab specifically binding to human transferrin receptor that has been fused to one of the C-termini of the heavy chains of the Abeta antibody via a peptidic linker.

For proper shuttling across the blood-brain-barrier the bispecific anti-Abeta/transferrin receptor antibody must be monovalent for the human transferrin receptor as the bivalent binding greatly reduces or even abolishes shuttling (see, e.g., WO 2016/207245, incorporated herein by reference).

As the bispecific anti-Abeta/transferrin receptor antibody comprises two different heavy chains the CH3 domains of the heavy chains have been modified according to the knobs-into-hole heterodimerization strategy (see Merchant et al. supra). The heavy chain conjugated to the additional Fab comprises the knob-mutations whereas the other, not extended, heavy chain comprises the hole mutations.

For this exemplary antibody, the antibody-related avid-binding side-product to be detected in the method according to the invention is a homodimer comprising two heavy chains conjugated to the additional Fab, i.e. an antibody comprising two binding sites to the Abeta protein and two binding sites the human transferrin receptor. The correctly assembled bispecific antibody comprises two binding sites to the human Abeta protein but only one binding site to the human transferrin receptor as outlined above. This antibody-related avid-binding side-product has to be detected as due to the bivalent binding to the human transferrin receptor proper shuttling across the blood-brain-barrier can no longer be effected.

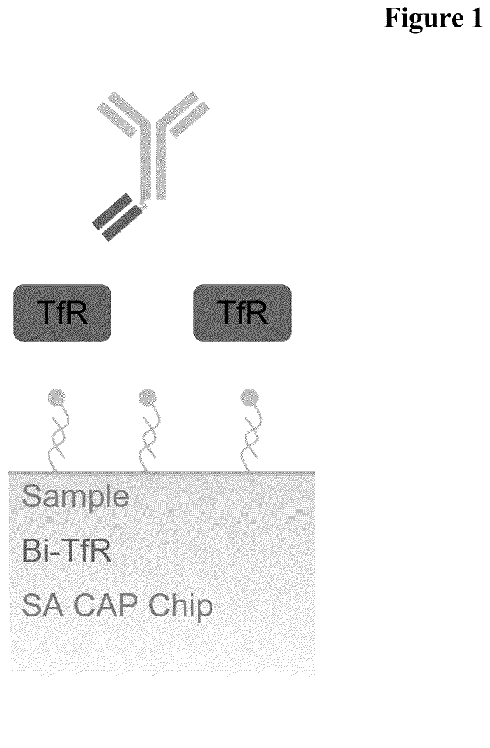

According to the method of the invention the antigen to which the side-product to be detected comprises an increased number of binding sites compared to the correctly assembled antibody, i.e. in this example two instead of one, has been immobilized on the surface of the SPR chip. In this example, the immobilization was an indirect immobilization. The antigen, i.e. the human transferrin receptor, has been conjugated to a biotin tag and captured on the surface of a streptavidin-modified CAP chip (Cytiva) after hybridizing the single-stranded DNA pre-coated on the chip with its streptavidin bearing complement strand (see FIG. 1).

The immobilization was done in this Example to about 2250 RU.

To the surface with the immobilized human transferrin receptor a sample comprising the correctly assembled bispecific anti-Abeta/transferrin receptor antibody was applied. Said correctly assembled antibody specifically binds to the immobilized human transferrin receptor, and becomes thereby bound to the chip surface. This generates an SPR signal. To dissolve the antibody from the chip surface a buffered aqueous solution not comprising the bispecific antibody or any other compound binding to human transferrin receptor was applied to the chip. The decay of the SPR (binding) signal was recorded and is the reference value.

For the determination of the presence/amount of the antibody-related side-product with avid-binding to the human transferrin receptor in the method according to the invention, an immobilized transferrin receptor chip surface has been prepared as outlined before. To this surface with the immobilized human transferrin receptor a sample comprising the correctly assembled antibody as well as the homodimeric antibody-related avid-binding side-product was applied. The antibody as well as the side-product bind to the immobilized human transferrin receptor and become thereby bound to the chip surface.

At low side-product concentrations, the weight difference of the correctly assembled antibody and the antibody-related homodimeric side-product is so small that substantially no detectable difference in the SPR signal occurs compared to the binding of only the correctly assembled antibody after applying the sample to the chip surface.

However, the antibody-related homodimeric side-product due to its bivalent and avid-binding to the immobilized transferrin receptor accumulates on the chip surface during the loading phase as it displaces correctly assembled antibody that is only monovalent for the immobilized transferrin receptor and has only affine binding. The complex of the avid-binding side-product with the transferrin receptor has a higher complex stability than the complex of the correctly assembled affine-binding antibody and thereby an accumulation thereof is achieved.

To dissolve the correctly assembled antibody as well as the avid-binding side-product from the chip surface a buffered aqueous solution not comprising said antibodies or any other compound binding to the human transferrin receptor was applied to the chip. The decay of the SPR (binding) signal was recorded and is the signal value.

At a defined time point after the start of the dissolving, the difference between the signal value and the reference value is determined. This difference is the termed residual binding.

The residual binding is depending on the amount of antibody-related homodimeric avid-binding side-product in the sample.

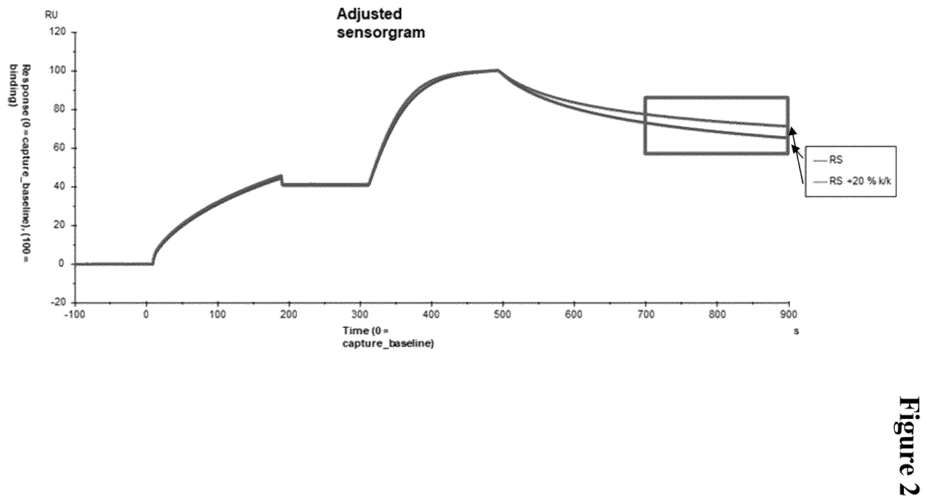

An exemplary SPR sensorgram is sown in FIG. 2 (RS=correctly assembled antibody; k/k=knob-knob-homodimer).

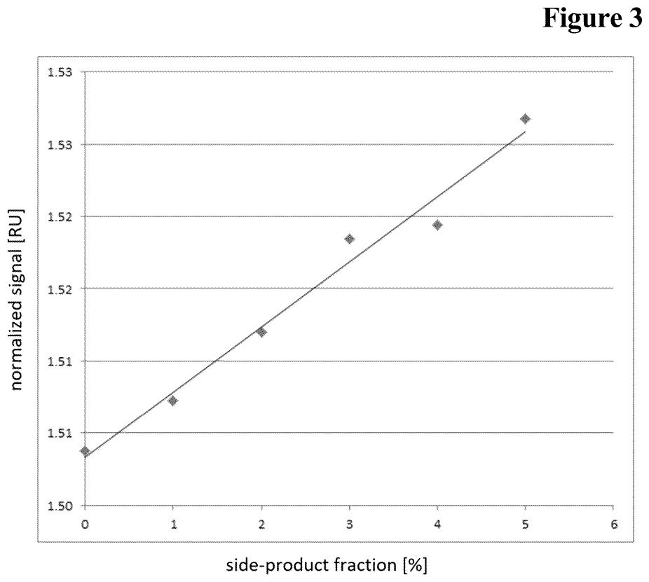

By spiking known amounts of antibody-related homodimeric avid-binding side-product into isolated correctly assembled antibody samples, different standards were prepared and a calibration curve was generated. The calibration curve is linear up to at least 5% (w/w) antibody-related homodimeric avid-binding side-product content (see FIG. 3) with an R² value of 0.9785.

Thus, the determined residual binding can be/is directly correlated to the amount of antibody-related homodimeric avid-binding side-product in the applied sample.

By the application of the method according to the invention it is now possible to quantify low amounts of antibody-related homodimeric avid-binding side-product in the sample. Especially the quantification of amounts well below 2% of the antibody-related avid-binding side-product is possible.

Conditions of this Example for an antibody in 2+1-format:
immobilization level of antigen=2250 RU
antibody concentration=100 nM
analyte flow rate=30 μl/min
association time=180 sec.
dissociation time=600 sec.
temperature=25° C.
buffer composition=HBS-EP+

It has to be pointed out that beside a sequential determination/recording of the reference value and the signal value this could be done in parallel using the different flow cells of an SPR chip or different SPR devices.

However, the standard referencing applied to, i.e. the subtracting of the results obtained with, a blank cell has to be considered. This is done to exclude the effect of any independent/unspecific binding.

The Method According to the Invention with a TCB-Format Antibody

This is a second Example of the method according to the invention. The method according to the invention is in the following exemplified with a bispecific anti-CD3/antigen-2 antibody. This is merely presented to exemplify the invention and shall not be construed as limitation. The true scope of the invention is set forth in the appended claims.

A bispecific anti-CD3/antigen-2 antibody is used in this example. This antibody is based on a Y-shaped full-length antibody comprising two Fabs (specifically) binding to antigen-2 and one additional monospecific Fab (specifically) binding to CD3 that has been inserted between one of the Fabs of the full-length antibody (specifically) binding to antigen-2 and the hinge region. This antibody format is called TCB (T-cell activating bispecific antibody format).

TCBs engage T-cells via their CD3 binding site that (specifically) binds to the T-cell receptor (TCR) complex on the surface of T-cells. For TCBs a homodimeric side-product comprising two CD3 binding sites poses a significant safety risk due to its ability to activate T-cells in the absence of target cells by TCR dimerization. This off-target (also known as target-cell-independent) T-cell activation can induce cytokine secretion or trigger an undesired immune response in patients. Off-target T-cell activation by an antibody-related homodimeric avid-binding side-product comprising two CD3 binding sites is an undesired activity and distinct from the intended T-cell activation by the correctly assembled TCB. Therefore, it is important to determine the amount of antibody-related homodimeric avid-binding side-product comprising two CD3 binding sites.

As the bispecific anti-CD3/antigen-2 antibody comprises two different heavy chains the CH3 domains of the heavy chains have been modified according to the knobs-into-hole heterodimerization strategy (see Merchant et al., supra). The extended heavy chain with the inserted additional Fab (specifically) binding to CD3 comprises the knob-mutations whereas the other not-extended heavy chain comprises the hole mutations.

For this exemplary antibody, the antibody-related avid-binding side-product to be detected in the method according to the current invention is a homodimer comprising two heavy chains each with inserted Fab, i.e. a compound comprising two binding sites to CD3. The correctly assembled bispecific antibody comprises only one binding site to CD3 as outlined above. This side-product has to be detected as due to the bivalent binding to CD3 it can effect off-target T-cell activation.

In line with the method according to the invention the antigen to which the side-product to be detected comprises an increased number of binding sites compared to the correctly assembled antibody, i.e. in this case two instead of one, has been immobilized on the surface of the SPR chip. In this example, the immobilization was a direct immobilization. The antigen, i.e. CD3, has been immobilized to the chip surface by amine coupling according to the manufacturer's instructions.

The immobilization was done to about 6900 RU of CD3 on a CM5 chip using amine coupling according to the manufacturer's instructions.

To the surface with the immobilized CD3 a sample comprising the correctly assembled bispecific antibody was applied. Said correctly assembled bispecific antibody binds to the immobilized CD3 and becomes thereby bound to the chip surface. This generates an SPR signal. To dissolve the bispecific antibody from the chip surface a buffered aqueous solution not comprising the bispecific antibody or any other compound binding to CD3 was applied to the chip. The decay of the SPR (binding) signal was recorded and is the reference value.

In the second part of the method, an immobilized CD3 chip surface has been prepared as outlined before. To this surface with the immobilized CD3 a sample comprising the correctly assembled bispecific antibody as well as the bispecific antibody-related homodimeric avid-binding side-product was applied. Both bind to the immobilized CD3 and become thereby bound to the chip surface.

The weight difference of the correctly assembled antibody and the antibody-related homodimeric avid-binding side-product is so small that substantially no detectable difference in the SPR signal occurs compared to only the binding of the correctly assembled antibody.

However, the homodimeric side-product due to its bivalent and avid binding to the immobilized CD3 accumulates on the chip surface. It replaces correctly assembled bispecific antibody that is only monovalent to the immobilized CD3 and that has only affine binding during the loading phase. The complex of the avid-bound bispecific antibody-related side-product has a higher complex stability compared to the complex of the correctly assembled affine-binding bispecific antibody and thereby accumulation during the loading phase is possible.

To dissolve the correctly assembled bispecific antibody as well as the avid-binding side-product from the chip surface a buffered aqueous solution not comprising said antibodies or any other compound binding to CD3 has been applied to the chip. The decay of the SPR (binding) signal was recorded and is the signal value.

At a defined time point after the start of the dissolving the difference between the signal value and the reference value is determined. This difference is the termed residual binding.

The residual binding is depending on the amount of bispecific antibody-related homodimeric avid-binding side-product in the sample.

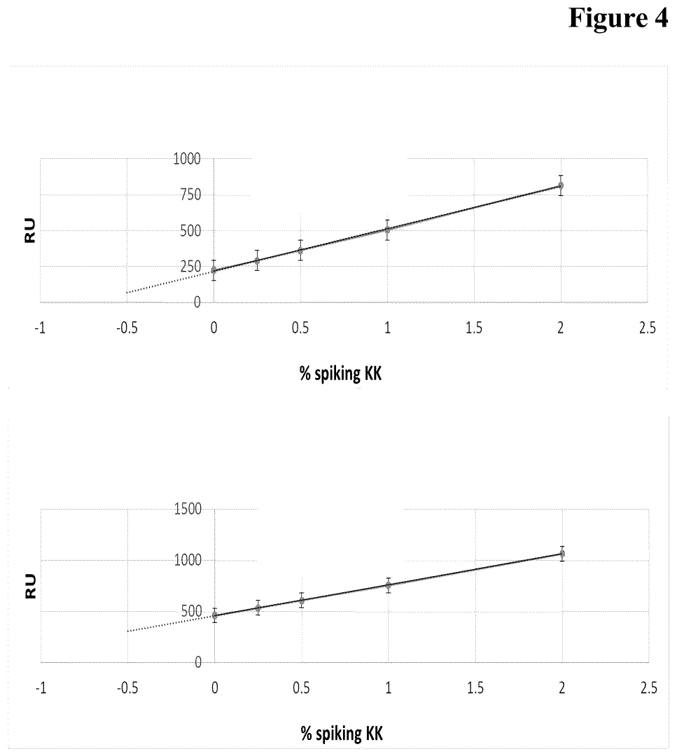

Two different approaches for the determination of the calibration curve have been used. In the first one (upper curve in FIG. 4) the spiking of avid-binding side-product (knob-knob homodimer; KK) was done to the isolated bispecific antibody, i.e. to a preparation containing no detectable amounts of avid-binding side-product (R² value of 0.9994). In the second one (lower curve in FIG. 4) the spiking of avid-binding side-product was done in reference material already comprising detectable amounts of the avid-binding side-product (R2 value of 0.9998). It can be seen that a calibration curve can also be established in this case. The calibration curve is linear at least until 2% (w/w) of the homodimeric side-product.

Thus, the determined residual binding can be correlated to an amount of bispecific antibody homodimeric avid-binding side-product in the applied sample.

By the application of the method according to the invention it is now possible to quantify low amounts of homodimeric side-product in the sample.

Summary of the conditions of this Example for an antibody in TCB format:
immobilization level of antigen=6900 RU
antibody concentration=100 nM
analyte flow rate=20 μl/min
association time=480 sec.
dissociation time=1200 sec.
temperature=37° C.
buffer composition=HBS-P+
   The Method According to the Invention with a CrossMab-Format Antibody

This is a further example of the method according to the invention. In this Example, the method according to the invention is exemplified with a bispecific antibody specifically binding to two different CD antigens. This is merely presented to exemplify the invention and shall not be construed as limitation. The true scope of the invention is set forth in the appended claims.

The bispecific antibody used in this example is a Y-shaped full-length antibody comprising one binding site specifically binding to the first CD antigen and one binding site specifically binding to the second, different CD antigen. The bispecific antibody is in the so-called CrossMab format (see supra).

As the bispecific antibody comprises two different heavy chains the CH3 domains of the heavy chains have been modified according to the knobs-into-hole heterodimerization strategy (see Merchant et al., supra). The heavy chain binding to the first CD antigen comprises the knob-mutations whereas the other heavy chain comprises the hole mutations.

For this exemplary antibody the antibody-related avid-binding side-product to be detected in the method according to the invention is a homodimer comprising two heavy chains both binding to the first CD antigen. The correctly assembled bispecific antibody comprises only one binding site for each CD antigen.

The bivalent binder to the first CD antigen has to be detected as due to the bivalent binding to the first CD antigen adverse, i.e. toxic side-effects, can be generated, e.g. by target-independent T-cell activation. Thus, monitoring and (precise) quantification of these antibody-related homodimeric avid-binding side-products is required as these side-products could be potential safety risks by target independent activation of T-Cells.

According to the method according to the invention the antigen to which the side-product to be detected comprises an increased number of binding sites compared to the correctly assembled antibody, i.e. two instead of one, has been immobilized on the surface of the SPR chip. In this example, the immobilization of the first CD antigen was a direct immobilization to the chip surface.

The immobilization of the first CD antigen was done to more than >2000 RU. However, it has been found that it should not exceed 4000 RU.

To the surface with the immobilized first CD antigen a sample comprising the correctly assembled antibody was applied. Said correctly assembled antibody specifically binds to the immobilized CD antigen and becomes thereby bound to the chip surface. This generates an SPR signal. To dissolve the antibody from the chip surface a buffered aqueous solution not comprising the bispecific antibody or any other compound binding to the first CD antigen has been applied to the chip. The decay of the SPR (binding) signal was recorded and is the reference value.

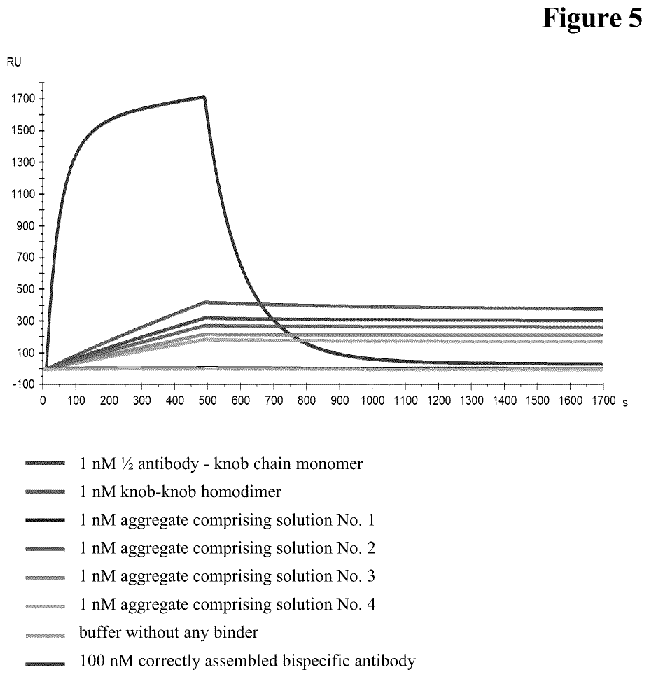

The different possible side-products with increased binding valency to the first CD antigen have also been applied to a chip as outlined above (see FIG. 5). It can be seen that all antibody-related avid-binding side-products (including apparently (K) knob monomer that actually spontaneously associates in solution to homodimers (confirmed by SEC) and (KK) knob-knob homodimers (covalent and non-covalent forms behave similar) and different aggregate comprising solutions) are retained on the surface due to more stable avid binding than the correctly assembled bispecific antibody.

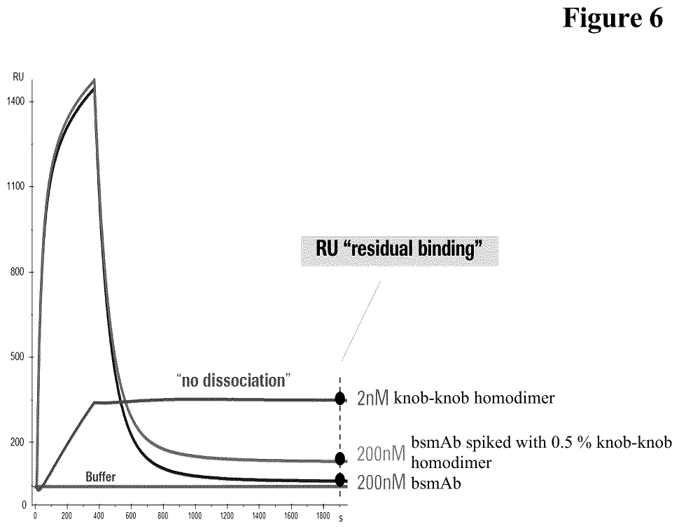

It can be seen that isolated avid-binding side-products do not dissociate with a detectable dissociation rate (see FIG. 6) whereas the correctly assembled bispecific antibody (bsmAb) does.

A long dissociation phase can be used in order to emphasize and more clearly exploit the difference in complex stability between the monovalent and the avid-binding antibody forms. Thereby the correctly assembled heterodimeric affine-binding bispecific antibody (monovalent first and second CD antigen binding) is dissolved from the chip surface. In contrast, all species with bi- and multivalent avid-binding interactions to the first CD antigen are retained. The SPR readout, i.e. the “residual binding”, can be determined after the long dissociation phase, e.g. after about 1200 seconds after start of the dissociation.

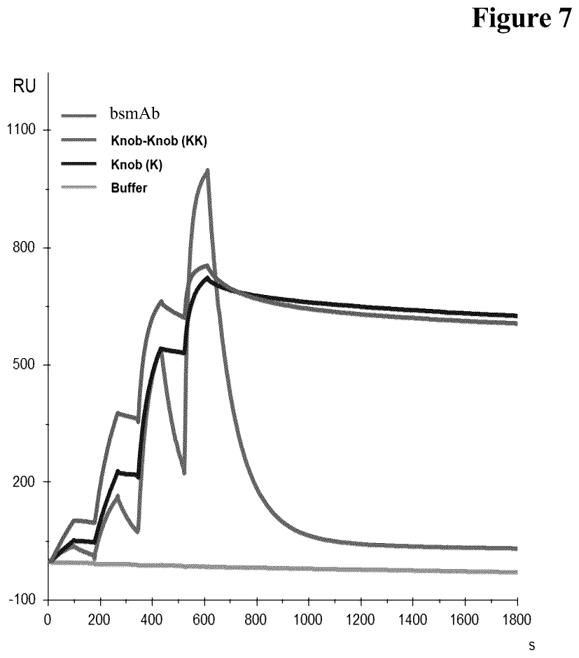

An immobilized first CD antigen chip surface has been prepared as outlined before. To this surface a sample comprising one isolated knob heavy chain (monomeric side-product) as well as the homodimeric side-product were applied. All antibodies (specifically) bind to the immobilized first CD antigen and become thereby bound to the chip surface (see FIGS. 6 and 7). It is assumed based on analytical data that the monomeric knob side-product dimerizes in situ and forms the homodimeric side-product.

The complex of the bound homodimeric avid-binding side-product has a higher complex stability than the complex of the correctly assembled heterodimeric affine-binding antibody and thereby an accumulation during the loading phase of this homodimeric avid-binding side-product is taking place.

To dissolve the correctly assembled antibody as well as the homodimeric avid-binding side product from the chip surface a buffered aqueous solution not comprising said antibodies or any other compound binding to the first CD antigen has been applied to the chip. The decay of the SPR (binding) signal was recorded. It can be seen that for the homodimeric avid-binding side-products a pronounced residual binding can be determined (see FIGS. 6 and 7).

In the second part of the method, an immobilized first CD antigen chip surface has been prepared as outlined before. To this surface with the immobilized first CD antigen a sample comprising solely the correctly assembled antibody as well as samples spiked with 1% (w/w) of the different antibody-related homodimeric avid-binding side-products was applied. All bind to the immobilized first CD antigen and become thereby bound to the chip surface.

The weight difference of the correctly assembled heterodimeric antibody and the homodimeric avid-binding side-product is too small to result in a detectable difference in the SPR signal.

Likewise, no difference can be detected using other biochemical methods like SEC, CE-SDS or HIC, as the molecular properties used for discrimination in these methods are the same. Only by the method according to the current invention a differentiation due to different kinetic binding properties can be achieved. As this difference is present in all heterodimeric antibody formats the method according to the current invention can be applied at least to any such format for the determination of antibody-related homodimeric avid-binding side-products.

The homodimeric side-product due to its bivalent and avid-binding to the immobilized first CD antigen accumulates on the chip surface. It replaces correctly assembled but only affine-binding bispecific antibody (only monovalent to the immobilized first CD antigen and therefore has only affine binding). The complex of the bound antibody-related homodimeric avid-binding side-product has a higher complex stability than the complex of the correctly assembled heterodimeric affine-binding bispecific antibody. Thereby accumulation of the side-product is possible.

To dissolve the correctly assembled heterodimeric affine-binding bispecific antibody as well as the antibody-related homodimeric avid-binding side-product from the chip surface a buffered aqueous solution not comprising said antibodies or any other compound binding to the first CD antigen has been applied to the chip. The decay of the SPR (binding) signal was recorded and is the signal value.

At a defined time point after the start of the dissolving the difference between the signal value and the reference value can be/is determined. This difference is the termed residual binding.

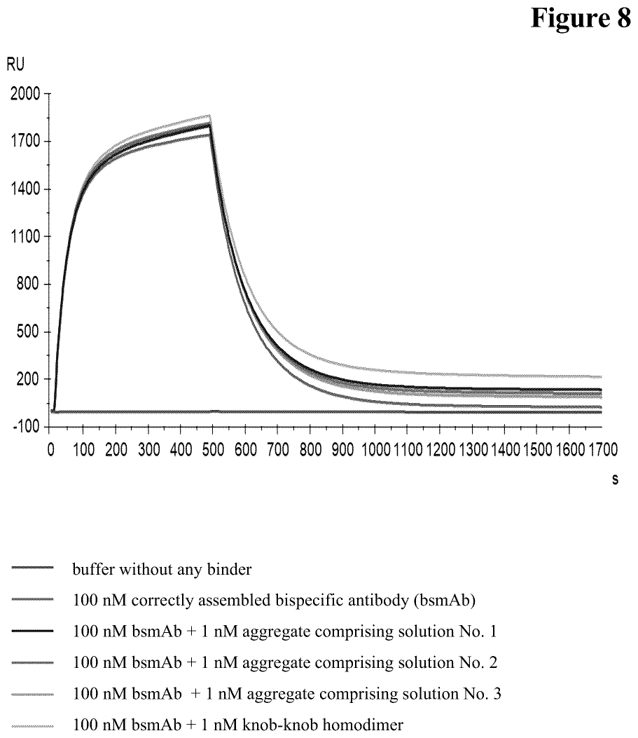

This effect is shown in FIG. 8, wherein 1% (w/w) of the avid-binding side-product has been added (1 nM side-product added to 100 nM correctly assembled antibody) to the isolated correctly assembled antibody (LS).

The residual binding difference is depending on the amount of homodimeric side-product in the sample.

All different antibody-related bi- and multivalent first CD antigen avid-binding side-products are quantified together, i.e. as a sum.

In case no isolated heterodimeric affine-binding bispecific antibody is available, the quantification can be performed by “calibration by standard addition”. A defined homodimeric molecule (homodimeric avid-binding side-product) is used as side-product standard. After the calculation, “% value of sum of highly active bivalent first CD antigen binders” is given as “% value equivalent of homodimeric standard”.

At a defined time point after the start of the dissolving the difference between the signal value and the reference value is determined. This difference is termed residual binding.

The residual binding difference is depending on the amount of homodimeric avid-binding side-product in the sample.

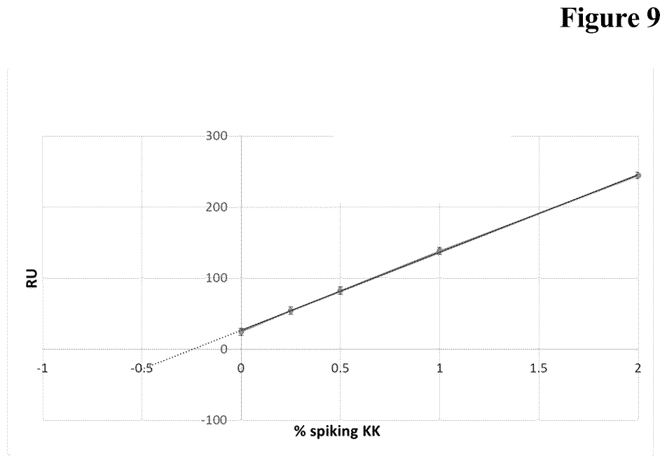

By spiking known amounts of homodimeric avid-binding side-product into isolated correctly assembled heterodimeric affine-binding antibody samples different standards were prepared and a calibration curve was generated. The calibration curve is linear up to at least 2% (w/w) homodimeric side-product content (R² value 0.9997) (see FIG. 9).

Thus, the determined residual binding can be correlated to the amount of homodimeric avid-binding side-product in the sample.

By the application of the method according to the invention it is now possible to quantify low amounts of homodimeric avid-binding side-product in the sample.

Conditions used in this Example for an antibody in CrossMab format:
immobilization level of antigen=2000 RU-4000 RU
antibody concentration=100 nM
analyte flow-rate=20 μl/min
association time=480 sec.
dissociation time=1200 sec.
temperature=25° C.
buffer composition=HBS-P+

### General Conditions for the Method According to the Invention:

antigen immobilization amount of at least about 2000 RU, in one preferred embodiment in the range of 2000 RU to 8000 RU
loading time in the range of 180 sec. to 480 sec., in one preferred embodiment about 360 sec.
concentration of the correctly-assembled antibody in the sample in the range of 50 nM to 200 nM, in one preferred embodiment about 100 nM
flow in the range of 10 μL/min to 30 μL/min, in one preferred embodiment in the range of about 20 μL/min to about 30 μL/min
residual binding can be determined at any time after the dissolving has started, in one preferred embodiment the residual binding is determined after at least 400 sec., after at least 480 sec., or after at least 1200 sec
no off-rate difference required between correctly assembled antibody and homodimeric side-product as the residual binding is dependent on the avidity of the side-product
residual binding can be adjusted via flow rate and times

### SPECIFIC EMBODIMENTS OF THE INVENTION

1. A method for determining avid-binding side-products of a bispecific antibody in a sample comprising correctly assembled bispecific antibody and one or more avid-binding side-product of the bispecific antibody using surface plasmon resonance,
   wherein the correctly assembled bispecific antibody comprises one or more binding site for a first antigen and one or more binding sites for a second antigen,
   wherein the avid-binding side-product of the bispecific antibody comprises two or more binding sites to the first antigen and more binding sites to the first antigen than the correctly assembled bispecific antibody,
   the method comprising the following steps:
      i) applying a loading solution comprising the correctly assembled bispecific antibody to an SPR surface on which the first antigen has been immobilized with at least 1000 RU to generate an SPR signal, applying thereafter a dissolution solution not comprising a compound binding to the first antigen to the SPR chip and recording the decay of the SPR signal,
      ii) applying a sample solution suspected to comprise an avid-binding side-product of the bispecific antibody to an SPR surface on which the first antigen has been immobilized with at least 1000 RU to generate an SPR signal, applying thereafter a dissolution solution not comprising a compound binding to the first antigen to the SPR chip and recording the decay of the SPR signal,
      iii) determining the avid-binding side-product of the bispecific antibody if the decay of the SPR signal determined in step ii) is slower than the decay of the SPR signal determined in step i).
2. The method according to embodiment 1, wherein the first antigen is immobilized with at least 2000 RU.
3. The method according to any one of embodiments 1 to 2, wherein the first antigen is immobilized with at least 2000 RU and at most 8000 RU.
4. The method according to any one of embodiments 1 to 3, wherein the applying of the loading solution and/or the sample is for at least 300 seconds at a flow rate of 5 μL/min to 50 μL/min.
5. The method according to any one of embodiments 1 to 4, wherein the applying of the loading solution and/or the sample is for at least 400 seconds at a flow rate of 5 μL/min to 50 μL/min.
6. The method according to any one of embodiments 1 to 5, wherein the applying of the dissolution solution is for at least 600 seconds at a flow rate of 5 μL/min to 50 μL/min.
7. The method according to any one of embodiments 1 to 6, wherein the applying of the dissolution solution is for at least 1200 seconds at a flow rate of 5 μL/min to 50 μL/min.
8. The method according to any one of embodiments 1 to 5, wherein the applying of the loading solution and the sample is under the same conditions.
9. The method according to any one of embodiments 1 to 3 and 6 to 8, wherein the applying of the dissolution solution in steps i) and ii) is under the same conditions.
10. The method according to any one of embodiments 4 to 9, wherein the flow rate is 15 μL/min to 35 μL/min.
11. The method according to any one of embodiments 1 to 10, wherein the determining in step iii) is with the SPR signal determined at a time point at least 400 sec. after the start of the application of the dissolution solution, and wherein the SPR signal obtained with the loading solution at the start of the application of the dissolution solution in step i) and ii) is made identical.
12. The method according to any one of embodiments 1 to 6 and 8 to 11, wherein the determining in step iii) is with the SPR signal determined at a time point at least 1200 sec. after the start of the application of the dissolution solution, and wherein the SPR signal obtained with the loading solution at the start of the application of the dissolution solution in step i) and ii) is made identical.
13. The method according to any one of embodiments 1 to 12, wherein the antibody is
   i) a bispecific antibody in CrossMab format, or
   ii) a bispecific antibody in TCB-format, or
   iii) a bispecific antibody in 2+1-format.
14. The method according to any one of embodiments 1 to 13, wherein the method is for determining the presence of an antibody-related homodimeric avid-binding side-product.
15. The method according to any one of embodiments 1 to 13, wherein the method is for quantifying the presence of an antibody-related homodimeric avid-binding side-product.

### FIGURES AND EXAMPLES

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Sketch of the assay setup with indirect immobilization of the human transferrin receptor via a biotin tag to the surface of a streptavidin-modified CAP chip after hybridizing the single-stranded DNA pre-coated on the chip with its streptavidin bearing complement strand.

FIG. 2 Decay of SPR signal during the dissolving of isolated correctly assembled antibody (lower curve; red) as well as a sample comprising 20% of an avid-binding side-product (upper curve; green) from the chip surface using a buffered aqueous solution not comprising said antibodies. The difference between the upper signal value and the lower signal value is the residual binding.

FIG. 3 Calibration curve for the determination of avid-binding side-product of a bispecific antibody in 2+1-format established with a method according to the invention.

FIG. 4 Calibration curves for the determination of avid-binding side-product of a bispecific antibody in TCB-format established with a method according to the invention. Upper curve: spiking of avid-binding side-product into isolated antibody; lower curve: spiking of avid-binding side-product in reference material already comprising avid-binding side-product.

FIG. 5 SPR sensorgrams of the isolated different possible side-products with increased binding valency to first CD antigen.

FIG. 6 SPR sensorgram showing the residual binding depending on the amount of avid-binding side-product.

FIG. 7 Dissolving of the correctly assembled antibody as well as the homodimeric avid-binding side product from the chip surface using a buffered aqueous solution not comprising said antibodies—the decay of the SPR (binding) signal was recorded—showing that for the homodimeric avid-binding side-products a pronounced residual binding can be determined.

FIG. 8 Decay of SPR signal for samples comprising 1% (w/w) of the avid-binding side-product (1 nM side-product added to 100 nM correctly assembled antibody).

FIG. 9 Calibration curve determined with the method according to the invention by spiking known amounts of homodimeric avid-binding side-product into isolated correctly assembled heterodimeric affine-binding antibody samples; different standards were prepared and a calibration curve was generated—the calibration curve is linear up to at least 2% (w/w) homodimeric side-product content.

### DESCRIPTION OF THE EXAMPLES

### Example 1

### Determination of Avid-Binding Side-Products Using a Method According to the Invention: 2+1-Format/Anti-Abeta/TfR Bispecific Antibody

A BIAcore CAP ship (Cytiva) was docked to a BIAcore T200 instrument and prepared as per instructions provided by the vendor. Then multiple assay cycles were performed. All cycles consisted of a hybridization step according to the manufacturer's instructions to bind the streptavidin on the chip surface. This was followed by an injection of a 1 μM biotinylated transferrin receptor solution for 180 sec. at a flow rate of 5 μl/min. Thereafter, the sample was injected into the flow chamber for 180 sec. (association phase) with a flow rate of 30 μl/min and subsequently dissociated from the transferrin receptor by switching to buffer for 600 sec. at the same flow rate. Finally, the chip surface was regenerated using the solutions included in the SA CAP kit.

In consecutive cycles performed as above, a range of antibody-antibody side-product mixtures (0%, 1%, 2%, 3%, 4%, 5%, 10%, 20%, 50% and 100% antibody side-product) were used as samples. Resulting signals were processed using the BIAcore T200 evaluation software. Using the “result plot” feature, capture levels were adjusted and a linear fit across the reported signals 10 sec. before the end of the dissociation phase was performed.

The assay was conducted at 25° C. with HBS-EP+ buffer as running and sample buffer (aqueous buffer solution containing 0.1 M HEPES, 1.5 M NaCl, 0.03 M EDTA, 0.5% (v/v) Surfactant P20, pH 7.4).

### Example 2

### Determination of Avid-Binding Side-Products Using a Method According to the Invention: TCB-Format

For the quantification of low amounts of bivalent CD3 binding homodimer side-products a BIAcore T200 was used. The CD3 antigen was immobilized by amine coupling to >6000 RU on a CM5 Chip (CD3 antigen stock solution was diluted to a concentration of 10 μg/mL in 10 mM sodium acetate buffer of pH 5.0). After the immobilization the analysis was performed at 37° C. and using HBS-P+ as running and dilution buffer (aqueous buffer solution containing 0.1 M HEPES, 1.5 M NaCl, 0.5% (v/v) Surfactant P20, pH 7.4). The sample to be analyzed was diluted to 100 nM antibody concentration. Calibration standards were prepared as follows: defined amounts of side-product were added to the 100 nM sample (e.g. 0.25%, 0.5%, 1%. 2% of knob-knob homodimeric (KK) species). The sample and the calibration standards were injected for 480 sec. over the CD3 derivatized chip surface. After a 1200 sec. dissociation time at a flow rate of 20 μl/min the regeneration of the surface followed by a 60 sec. regeneration step with a 3 M MgCl₂ solution. The data was analyzed using the BiaEvaluation software using the concentration analysis using Calibration' functionality. Calibration settings are specified as follows: Flow Cell: FC2-1 or FC4-3, Report Point: stability at 1200 sec., Response Type: Relative Response, Fitting Function: Linear. Quantification is done by the means of calibration by standard addition. The % value of homodimer CD3: equivalent of KK=Intercept/slope.

### Example 3

### Determination of Avid-Binding Side-Products Using a Method According to the Invention: CrossMab Format

For the quantification of low amounts of the first CD antigen binding homodimeric side-products a BIAcore T200 was used. The first CD antigen was immobilized at a level between 2000 RU and 4000 RU on an SA Chip at a flow rate of 10 μL/min (first CD antigen stock solution was diluted to concentration of 5 μg/mL in HBS-P+buffer). After the immobilization the analysis was performed at 25° C. with HBS-P+ as running and dilution buffer. The antibody sample to be analyzed was diluted to 100 nM. In parallel, several samples were prepared for calibration by standard addition: defined amounts of side-product were added to the 100 nM antibody sample (e.g. 0.25%, 0.5%, 1% and 2% of knob-knob homodimeric (KK) side-product). The antibody sample and the calibration samples were injected for 480 sec. at a flow of 20 μL/min over the first CD antigen comprising chip surface. After the 1200 sec. dissociation time at a flow rate of 20 μl/min, the surface was regenerated for 120 sec. with “Gentle Ag/Ab Elution Buffer pH 6.6” (Thermo Cat #21013). Data analysis: The data was analyzed using the BiaEvaluation software using the concentration analysis using Calibration' functionality. Calibration settings are specified as follows: Flow Cell: FC2-1 or FC4-3, Report Point: stability at 1200 sec., Response Type: Relative Response, Fitting Function: Linear. Quantification is done by the means of calibration by standard addition. The % value of homodimer KK first CD antigen: equivalent of KK=Intercept/slope.

1. A method for determining avid-binding side-products of a bispecific antibody in a sample comprising correctly assembled bispecific antibody and one or more avid-binding side-product of the bispecific antibody using surface plasmon resonance,
wherein the correctly assembled bispecific antibody comprises one or more binding site for a first antigen and one or more binding sites for a second antigen,
wherein the avid-binding side-product of the bispecific antibody comprises two or more binding sites to the first antigen and more binding sites to the first antigen than the correctly assembled bispecific antibody,
the method comprising the following steps:
i) applying a loading solution comprising the correctly assembled bispecific antibody to an SPR surface on which the first antigen has been immobilized with at least 1000 RU to generate an SPR signal, applying thereafter a dissolution solution not comprising a compound binding to the first antigen to the SPR chip and recording the decay of the SPR signal,
ii) applying a sample solution suspected to comprise an avid-binding side-product of the bispecific antibody to an SPR surface on which the first antigen has been immobilized with at least 1000 RU to generate an SPR signal, applying thereafter a dissolution solution not comprising a compound binding to the first antigen to the SPR chip and recording the decay of the SPR signal,
iii) determining the avid-binding side-product of the bispecific antibody if the decay of the SPR signal determined in step ii) is slower than the decay of the SPR signal determined in step i).

2. The method according to claim 1, wherein the first antigen is immobilized with at least 2000 RU.

3. The method according to claim 1, wherein the first antigen is immobilized with at least 2000 RU and at most 8000 RU.

4. The method according to claim 1, wherein the applying of the loading solution and/or the sample is for at least 300 seconds at a flow rate of 5 μL/min to 50 μL/min.

5. The method according to claim 1, wherein the applying of the loading solution and/or the sample is for at least 400 seconds at a flow rate of 5 μL/min to 50 μL/min.

6. The method according to claim 1, wherein the applying of the dissolution solution is for at least 600 seconds at a flow rate of 5 μL/min to 50 μL/min.

7. The method according to claim 1, wherein the applying of the dissolution solution is for at least 1200 seconds at a flow rate of 5 μL/min to 50 μL/min.

8. The method according to claim 1, wherein the applying of the loading solution and the sample is under the same conditions.

9. The method according to claim 1, wherein the applying of the dissolution solution in steps i) and ii) is under the same conditions.

10. The method according to claim 4, wherein the flow rate is 15 μL/min to 35 μL/min.

11. The method according to claim 1, wherein the determining in step iii) is with the SPR signal determined at a time point at least 400 sec. after the start of the application of the dissolution solution, and wherein the SPR signal obtained with the loading solution at the start of the application of the dissolution solution in step i) and ii) is made identical.

12. The method according to claim 1, wherein the determining in step iii) is with the SPR signal determined at a time point at least 1200 sec. after the start of the application of the dissolution solution, and wherein the SPR signal obtained with the loading solution at the start of the application of the dissolution solution in step i) and ii) is made identical.

13. The method according to claim 1, wherein the antibody is
i) a bispecific antibody in CrossMab format, or
ii) a bispecific antibody in TCB-format, or
iii) a bispecific antibody in 2+1-format.

14. The method according to claim 1, wherein the method is for determining the presence of an antibody-related homodimeric avid-binding side-product.

15. The method according to claim 1, wherein the method is for quantifying the presence of an antibody-related homodimeric avid-binding side-product.
